(19) United States
(12) Patent Application Publication
(10) Pub. No.: US 2021/0104322 A1
(43) Pub. Date: Apr. 8, 2021

(54) PERSONAL WELLNESS RECOMMENDATION ENGINE

(71) Applicant: GATC Health Corp, Irvine, CA (US)

(72) Inventors: Vaishnavi Narayan, Lehi, UT (US); Christopher Dean, North Ogden, UT (US); Ian Jenkins, Cedar Hills, UT (US)

(21) Appl. No.: 16/938,791

(22) Filed: Jul. 24, 2020

Related U.S. Application Data
(60) Provisional application No. 62/877,838, filed on Jul. 24, 2019.

(51) Int. Cl. G16H 50/20; G16H 10/40; G16H 10/60; G16H 50/30; G16H 50/70; G16H 20/60; G06N 3/04; G06N 3/08; G16B 40/30; G16B 20/20

(52) U.S. Cl. CPC G16H 50/20; G16H 10/40; G16H 10/60; G16H 50/30; G16H 50/70; G16H 10/20; G06N 3/04; G06N 3/088; G16B 40/30; G16B 20/20; G16H 20/60

(57) ABSTRACT

The disclosure generally describes computer-implemented methods, software, and systems for generating a health recommendation. A computer-implemented method includes receiving genetic data and user data of a user. The genetic data is processed to generate vector representations of a fixed size by using an unsupervised learning algorithm. Health risk features are extracted from the vector representations by using a multi-layer convolutional neural network. Dietary conditions associated with the health risk features are determined and used to generate the health recommendation. The health recommendation is displayed for the user.

### BACKGROUND

Many environmental factors, such as food, lifestyle, and stress can cause epigenetic changes by altering the way molecules bind to DNA. In some cases, environmental factors affect the way a DNA wraps around a protein. As a result of the changes, a gene behavior can be modified. For example, dramatic changes can occur by switching genes off when they should be on or vice-versa. Matching environmental factors to an individual's genetic profile can be useful for risk identification.

### SUMMARY

Implementations of the present disclosure include computer-implemented methods and systems configured for generating a health recommendation. In accordance with one aspect of the present invention, actions include receiving genetic data and user data of a user, processing the genetic data to generate vector representations of a fixed size by using an unsupervised learning algorithm, extracting health risk features from the vector representations by using a multi-layer convolutional neural network, determining dietary conditions associated with the health risk features, generating the recipe recommendation based on the dietary conditions and the user data, and displaying the recipe recommendation for the user. In some implementations, the genetic data includes reference numbers of single nucleotide polymorphisms, risk alleles, and genetic variants. In some implementations, the user data includes one or more of dietary restrictions, a panoramic profile, available ingredients, user preferences, and user recipe rating. In some implementations, the panoramic profile includes a metabolic profile based on at least one of a noninvasive measurement and an invasive measurement. In some implementations, the method of claim 4, wherein girth, a systolic blood pressure, and a diastolic blood pressure. In some implementations, the invasive measurement includes at least one of LDL cholesterol, HDL cholesterol, triglycerides (mg/dL), and blood glucose level (fasting blood sugar, mM). In some implementations, the unsupervised learning algorithm is characterized by a model for distributed word representation configured to automatically generate linear structures of the vector representations. In some implementations, the unsupervised learning algorithm is configured to perform operations including: processing the genetic data to generate groups of term co-occurrence, formatting the groups to generate a matrix, setting soft constraints for each entry of the matrix, adding a weight to each entry of the matrix, and generating the vector representations based on the matrix. In some implementations, generating the groups of term co-occurrence includes identifying a plurality of portions of the genetic data and determining a frequency of each portion of the genetic data. In some implementations, the weight prevents learning only from a limited subset of the plurality of portions of the genetic data that is most common. In some implementations, processing the genetic data to generate the vector representations of the fixed size includes: classifying the vector representations to generate a plurality of clusters, each cluster including one or more data points, determining a first distance between each data point and a center of each cluster, assigning the one or more data points to one of the plurality of clusters based on the first distance, determining a new cluster center for each cluster, determining a second distance between each data point and the new center of each cluster, and determining whether any data point is reassigned to a different cluster. In some implementations, the multi-layer convolutional neural network includes a plurality of layers including a convolution layer configured to extract health risk features from the vector representations and at least one of a rectified linear unit layer configured to add non-linearity to the health risk features, a pooling layer configured to down sample the health risk features, a dropout layer configured to filter out the noise of the health risk features, and a fully connected layer configured to determine patterns within the health risk features. In some implementations, the convolution layer includes a sliding window function that is applied to the vector representations of the fixed size to generate convolved features that are processed to extract the health risk features. In some implementations, the rectified linear unit layer is configured to process the health risk features generated by any of the plurality of layers that are identified as being linear. In some implementations, the pooling layer includes a filter configured to determine a maximum value of the health risk features generated by any of the plurality of layers that are within a window size. In some implementations, the dropout layer includes a noise filter configured to remove a portion of the health risk features generated by any of the plurality of layers that are associated to errors. In some implementations, the fully connected layer includes a connecting function configured to add a weight to each pair connection of the health risk features generated by any of the plurality of layers. In some implementations, generating a recipe recommendation includes identifying a set of users who previously rated recipes similar to the user and who have rated one of a plurality of recipes matching the dietary conditions of the user. In some implementations, the plurality of recipes is classified based on weighted average rankings. In some implementations, the set of users who previously rated recipes similar to the user is determined based on calculating cosine similarity for users with the dietary conditions. In some implementations, generating a recipe recommendation includes an analysis of an impact of past rated recipes on the user data. In some implementations, generating a recipe recommendation includes using a food-pairing network to predict ingredient combinations that match with each other.

The details of one or more implementations of the subject matter of the present disclosure are set forth in the accompanying drawings and the description below. Other features and advantages of the present disclosure will be apparent from the description and drawings, and from the claims.

### DESCRIPTION OF DRAWINGS

FIG. 1 depicts an example of an architecture that can be used to execute implementations of the present disclosure.

FIG. 2 depicts an example of a component architecture in accordance with implementations of the present disclosure.

FIG. 3 depicts an example of a component architecture in accordance with implementations of the present disclosure.

FIG. 4 is a flowchart illustrating an example of a method for generating a health recommendation, according to an implementation of the present disclosure.

FIG. 5 is a block diagram illustrating an example of a computer system used to provide computational functionalities associated with described algorithms, methods, functions, processes, flows, and procedures, according to an implementation of the present disclosure.

Like reference numbers and designations in the various drawings indicate like elements.

### DETAILED DESCRIPTION

Implementations of the present disclosure are generally directed to a genetic-based health risk identification to determine the health recommendations. More particularly, implementations of the present disclosure are directed to matching genetic information, dietary restrictions, and user preferences to provide an accurate recipe recommendation to support a user's health.

Implementations can include actions of receiving genetic data and user data of a user, processing the genetic data to generate vector representations of a fixed size by using an unsupervised learning algorithm, extracting health risk features from the vector representations by using a multi-layer convolutional neural network, determining dietary conditions associated with the health risk features, generating the recipe recommendation based on the dietary conditions and the user data, and displaying the recipe recommendation for the user. Risk identification models can utilize user data in order to determine a health risk using the provided user data. Given that genetic data, health conditions, and user preferences can be provided in different formats and form very large data sets, matching health risks with optimal health recommendations can be a resource-intensive process that can generate unwanted errors. Implementations of the present disclosure address this challenge. For example, in accordance with implementations of the present disclosure, in order to increase the accuracy of health risk identification, the user data can be processed by a machine-learning algorithm that can be reliably extract individual information using similarities between users.

The following detailed description is presented to enable any person skilled in the art to make and use the disclosed subject matter in the context of one or more particular implementations. Various modifications, alterations, and permutations of the disclosed implementations can be made and will be readily apparent to those or ordinary skill in the art, and the general principles defined can be applied to other implementations and applications, without departing from the scope of the present disclosure. In some instances, details unnecessary to obtain an understanding of the described subject matter can be omitted so as to not obscure one or more described implementations with unnecessary detail and inasmuch as such details are within the skill of one of ordinary skill in the art. The present disclosure is not intended to be limited to the described or illustrated implementations, but to be accorded the widest scope consistent with the described principles and features.

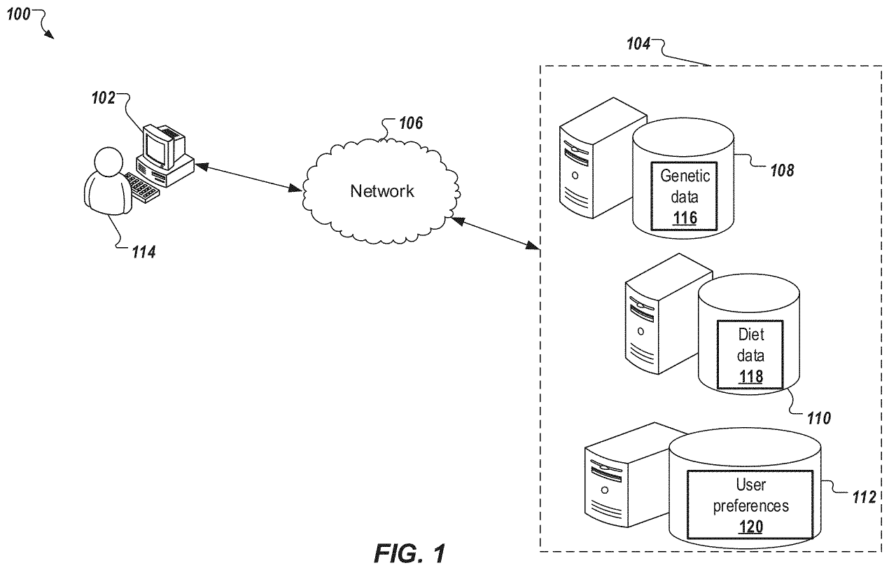

FIG. 1 depicts an example of an architecture 100 that can be used to execute implementations of the present disclosure. In the depicted example, the example architecture 100 includes one or more client devices 102, a server system 104 and a network 106. The server system 104 includes one or more server devices 108, 110, 112. In the depicted example, a user 114 interacts with the client device 102. In an example context, the user 114 can include a user, who interacts with a software application (or “application”) that is hosted by the server system 104.

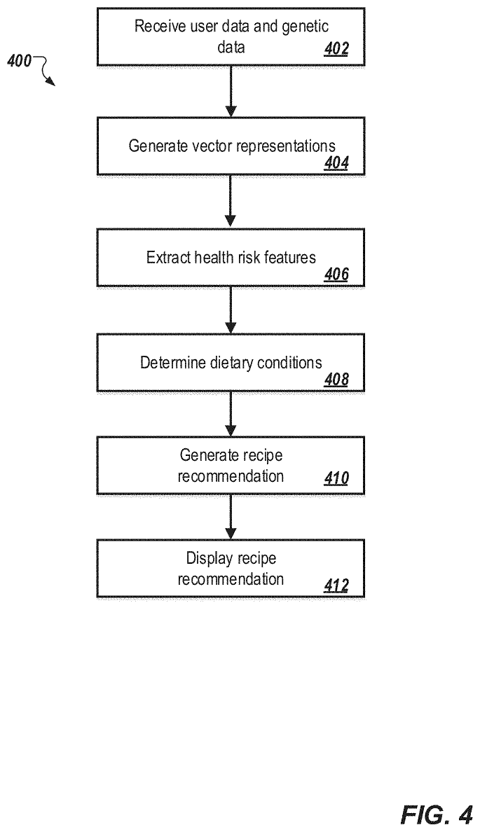

In some examples, the client device 102 can communicate with one or more of the server devices 108, 110, 112 over the network 106. In some implementations, the analytical web application processes user data (for example, genetic information 116, diet information 118, and user preferences 120) received from a database or a webpage, or a combination of database and webpage, processes the user data, and generates a health recommendation including recipe recommendations, before sending the health recommendation from the server system 104 to the user 114 over the network 106. The application driver, such as a standard component of the client device 102 can be configured to perform the operations described with reference to FIG. 4.

In some implementations, each server device 108, 110, 112 includes at least one server and at least one data store that stores user data (such as, genetic information 116, diet information 118, and user preferences 120). In some implementations, the server system 104 can be provided by a third-party service provider, which stores and provides access to user data including genetic information 116, diet information 118, user preferences 120 and other. In the example depicted in FIG. 1, the server devices 108, 110, 112 are intended to represent various forms of servers including, but not limited to, a web server, an application server, a proxy server, a network server, or a server pool. In general, server systems accept requests for application services (such as, health recommendation services) and provides such services to any number of client devices (for example, the client device 102) over the network 106.

In accordance with implementations of the present disclosure, the server system 104 can host a health recommendation algorithm (for example, provided as one or more computer-executable programs executed by one or more computing devices) that can be applied before completing a service request. For example, input data including genetic information, diet information and preference matching results can be provided to the server system (for example, from the client device 102), and the server system 104 can process the input data to provide result data. For example, the server system 104 can send the result data to the client device 102 over the network 106 for display to the user 114.

Implementations of the present disclosure function largely independently of the browser, and do not require any modification to the browser (for example, no browser extensions or plug-ins). Further, implementations of the present disclosure support more operations than a simple search, such as filtering, resampling, matching, and identification, which are typical operations used in analytical applications. Implementations of the present disclosure also provide a back-end computing system with risk results associated to a user at a point in time in order to support personalized health recommendations.

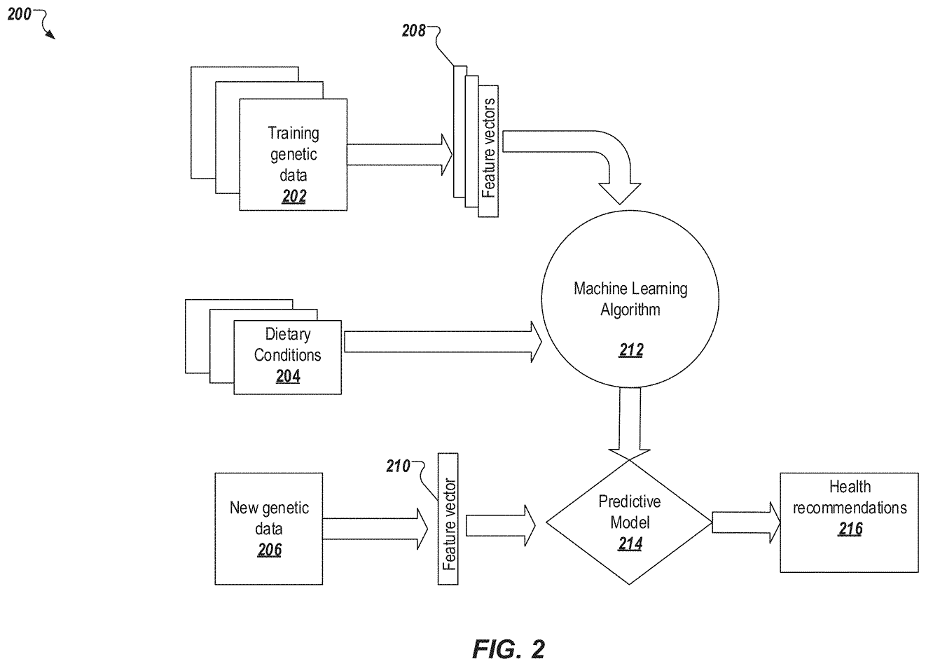

FIG. 2 depicts an example of a learning algorithm architecture 200 in accordance with implementations of the present disclosure. In some implementations, inputs of the example architecture 200 include training genetic data 202, dietary conditions 206, and new genetic data 208. The training genetic data 202 and the new genetic data 208 can be vectorized in training feature vectors 208 and new feature vectors 210, respectively, which can be processed by the processing components of the example architecture 200. The example architecture 200 can include as processing components a machine learning algorithm 212 and a predictive model 214. In some implementations, the machine-learning algorithm 212 and the predictive model 214 can be included in different servers. For example, the machine-learning algorithm 212 can be provided across multiple client-side application servers or on a server-side back-end computing system. The predictive model 214 can be provided on a client-side application server.

As shown in FIG. 2, the machine-learning algorithm 212 is configured to process vectorized training genetic data and a variety of dietary conditions to identify health recommendations based on dietary restrictions and health risk matching. The machine-learning algorithm 212 can include a communication manager that enables communication between the machine-learning algorithm 212 and the predictive model 214, over the network 106 (for example) using one or more processors of the client device 102 and the server 104.

The predictive model 214 can be configured to receive vectorized new genetic data and associations between dietary restrictions and health risks from the machine-learning algorithm 212, to determine health recommendations 216. The predictive model 214 can be configured to permit adjustment of results based on multiple conditions. The adjustment conditions include similarities of a variety of user profiles, flavor matching, cuisine identification and others. The new genetic data, user preferences, and/or new dietary restrictions may be created and modified by a user of a service server using a profiling framework of the predictive model 214. The predictive model 214 can be configured to transmit the health recommendations 216 to the client device 102.

Implementations of the present disclosure are designed to increase the accuracy of health recommendations based on user data, while minimizing processing costs. In particular, the components of the example architecture 200 have been configured to enable identification of health risks associated with a genetic profile prior to searching for recipes matching user preferences. Integrating health risk identification prior to searching for recipes and dynamic adjustments to dietary restrictions or new health conditions increases the accuracy of the personalized health recommendations.

In some implementations, a health recommendation includes a list of identified genes and for each gene in the list of genes, the corresponding RS, genotype, possible implications or traits, diet or recipe recommendations, foods to avoid, fitness recommendations, lifestyle choices to avoid, and recommended supplements. In some implementations, the health recommendation can be provided as one or more tables, having a format as the examples included in Tables 1A and 1B. The health recommendation can include links to additional documents (e.g., videos, webpages, or tables) that provide associated information (e.g., examples of recipes, examples of exercise types and schedules, and examples of supplements).

TABLE 1A
Depicts an example of a personalized health recommendation.

| | | Geno- | | | |
|---|---|---|---|---|---|
| Gene | RS | type | Implications/Traits | Diet/Recipe | Foods to Avoid |
| COMT | rs4680 | AA | The (“Worrier”) advantage is in | Eat foods high in boron | Processed foods |
| V158M | | | memory and attention to tasks | like chickpeas, almonds, | and refined |
| | | | More exploratory, lower COMT | beans, vegetables, | sugars. |
| | | | enzymatic activity, therefore | bananas, walnuts and | |
| | | | higher dopamine levels; lower | avocados. Eat foods high | |
| | | | pain threshold, enhanced | in magnesium (mg) like | |
| | | | vulnerability to stress, yet also | cooked spinach, Swiss | |
| | | | more efficient at processing | chard, dark chocolate, | |
| | | | information | dried pumpkin seeds, | |
| | | | | almonds, avocados, | |
| | | | | almonds, and figs. | |
| COMT | rs4680 | AG | Intermediate dopamine levels, | Eat a healthy diet of | Processed foods |
| V158M | | | other effects, complex. | unprocessed food, | or refined |
| | | | | minimize added sugar, | foods. |
| | | | | and drink plenty of | |
| | | | | water. | |
| COMT | rs4680 | GG | The (“Warrior”) has multiple | Food on Omega-3 fatty | Processed foods |
| V158M | | | associations. Val, less exploratory, | acids, which can be | or refined |
| | | | higher COMT enzymatic activity, | found in cold-water fish, | foods. |
| | | | therefore lower dopamine levels; | nuts, avocados, olive oil, | |
| | | | higher pain threshold, better | and sunflower seeds. | |
| | | | stress resiliency, albeit with a | Drink plenty of water. | |
| | | | modest reduction in executive | | |
| | | | cognition performance under | | |
| | | | most conditions. | | |

TABLE 1B
Depicts another example of a personalized health recommendation.

| | Fitness | Fitness | Lifestyle | Lifestyle | |
|---|---|---|---|---|---|
| Gene | Recommendation | Warnings | Recommendation | Warnings | Supplements |
| COMT | Do aerobic | High | Monitor and | Avoid alcohol | Boron, I3C[Indole 3 |
| V158M | exercise at or | intensity | reduce stress. | and tobacco. | Carbon], Fish Oil [Omega- |
| | below target | training or | Reduce exposure | Limit exposure | 3], Glutathione, |
| | heart rate. | exercise at | to amphetamines | to plastic | Magnesium (M), |
| | | max VOC. | Consider adding | bottles. Avoid | Phosphatidylserine, High |
| | | | calming essential | microwaving | Quality B Vitamin Complex |
| | | | oils. Exercise | foods in plastic | Vitamin B1 (Thiamine **) |
| | | | caution with | containers. | Vitamin B2 (Riboflavin), |
| | | | dopamine - | Avoid caffeine | Vitamin B3 (Niacin/ |
| | | | increasing drugs. | that can | niacinamide), Vitamin B5 |
| | | | Speak with your | increase | (Pantothenate), Vitamin B6 |
| | | | physician about | cortisol levels | [pyridoxal 5 phosphate |
| | | | any diet, lifestyle | and anxiety. | (PLP)], Vitamin B9 (Folic |
| | | | or supplement | | Acid: Quatrefolic Acid and |
| | | | changes. | | 5-Methyltetrahydrofolate) |
| | | | | | Vitamin B 12 |
| | | | | | (Methylcobalamin), |
| | | | | | Vitamin A (Beta-Carotene), |
| | | | | | Vitamin C (Ascorbic Acid) |
| | | | | | Vitamin E |
| COMT | | | | | |
| V158M | | | | | |
| COMT | High Intensity | | | | L-Dopa |
| V158M | interval training | | | | |
| | (HIIT) exercises. | | | | |

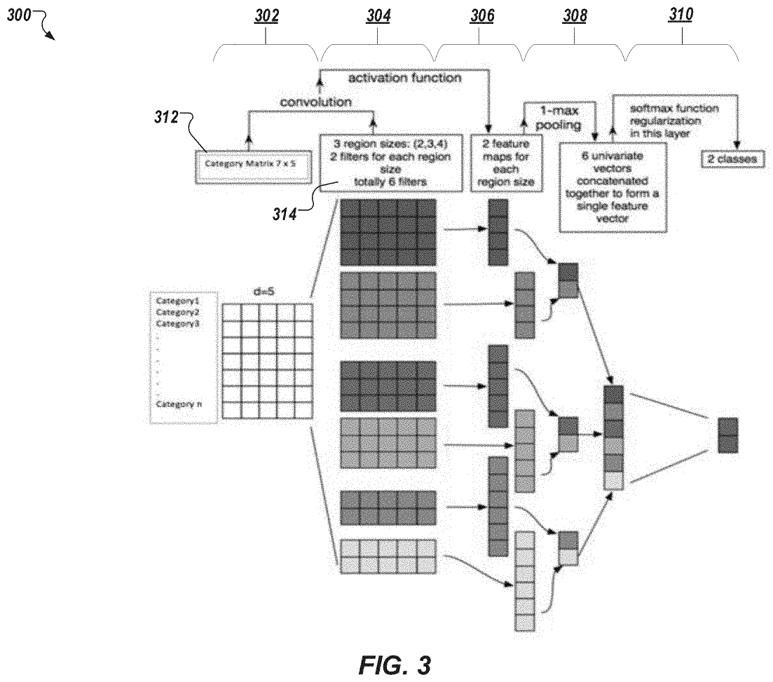

FIG. 3 is an example architecture of a convolutional neural network (CNN) 300. CNN 300 is a class of feed-forward-artificial-neural-network. CNN 300 contains several different layers, including a convolution layer 302, a rectified linear unit layer 304, a pooling layer 306, a dropout layer 308, and a fully connected layer 310. The CNN 300 can be designed to take maximum advantage of the 2D inputs (matrices 312) by using associated weights and local connections processed by a pooling function, such as softmax.

The convolution layer 302 is configured to perform a convolution function (a sliding window function) that is applied to a matrix 312. The matrix 312 can include an input text or image. Each entry in the matrix represents a part of the input data. The matrix 312 can be processed using a sliding window called the kernel or the feature detector using a convolution function. The convolution function includes an integral of a product of two functions over an infinite range. One of the functions is reversed and shifted before integrating the result.

$$\begin{matrix}( f * g ) ( t ) {=}^{def} {\int }_{- ∞}^{∞} f ( τ ) g ( t - τ ) dτ \\ = {\int }_{- ∞}^{∞} f ( t - τ ) g ( τ ) dτ ,\end{matrix} \qquad ( 1 )$$

where ƒ(τ) is the weighted average of t and g(−τ) is the weighting shifted by amount t. With every change in t, the weighting function emphasizes different parts of the input matrix.

Filters 314 that slide over patches of the matrix or over entire rows of matrices can be used. The width of the filters 314 can be the same as the width of the input matrix 312.

The rectified linear units (ReLU) layer 304 (also called an activation layer) can be configured to introduce non-linearity in the CNN 300. Using the ReLU layer 304 increases the training speed of CNN 300. The ReLU layer 304 is based on the following equation:

ƒ(x)=max(0, x), (2)

where f(x) is the function that is applied to all the values in the input volume. In some implementations, CNN 300 includes multiple ReLU layers 304. For example, a ReLU layer 304 can be used after each other layer that introduces linearity.

The pooling layer 306 includes a sample-based-discretization process. The pooling layer 306 is also called a down-sampling layer. The pooling layer 306 down samples the input representation. The pooling layer 306 is configured to enable better assumptions about the features contained in the sub-regions of a filtered matrix 312. Max pooling can be performed by applying a max filter to non-overlapping sub-regions. Max pooling determines the max value for every sub-region that is represented by the filter. The filter size in this example is 2×2. The max value of every sub-region is obtained forming another matrix. As a result, max pooling reduces the number of parameters within the model and generalizes the results of the convolutional filter.

The dropout layer 308 can be configured to eliminate the noise in the data is captured by the network that can be leading to an overfitting problem. Overfitting problem occurs if the model has a low bias and a high variance. Bias in machine learning can be the result of erroneous assumptions by the learning algorithm 212. Variance can be the result of sensitivity to small changes in the training data. Overfitting, can be a result of a highly complex and intricate model. Dropout is a technique of randomly ignoring some set of selected neurons during training. For example, some neurons are identified as being incapable of forward pass or weight management. As a neural network learns, neuron weights sink into their setting inside the system. If neurons are randomly dropped from the network, other neighboring neurons would have to step-in and handle the falsified neurons, thereby making better predictions. The dropout layer 308 can provide a better generalization of the training data.

In the fully connected layer 310, each neuron is connected to every neuron from the previous layer. Every connection has its own weight. The fully connected layer 310 provides a general-purpose connection pattern and makes no assumptions about the features in the data. The fully connected layer 310 enables a control of memory load requirements (weights) and computation costs (connections). Though the neurons are connected to every neuron from the previous layer in this layer, the neurons in the same layer are not connected. The results of the AI can be labelled data that would be fed into the front-end of the framework. The results can include the genotype with the risk associated with it. For example, for RS4986883—Risk Allele—AA: the user can be at a particular determined percentage of a health risk (e.g., cardiovascular related diseases).

FIG. 4 is a flowchart illustrating an example of a method 400 for generating a health recommendation, according to an implementation of the present disclosure. Method 400 can be implemented as one or more computer-executable programs executed using one or more computing devices, as described with reference to FIGS. 1-3. In some implementations, various steps of the example method 400 can be run in parallel, in combination, in loops, or in any order.

At 402, a user input is received by a computing device. The user input can include user data and genetic data. In some examples, the genetic data includes reference numbers of single nucleotide polymorphisms, risk alleles, and genetic variants. In some examples, the user data includes one or more of dietary restrictions, a panoramic profile, available ingredients, user preferences, and user recipe rating. The panoramic profile can include a metabolic profile based on at least one of a noninvasive measurement and an invasive measurement. The noninvasive measurement can include at least one of a gender, an ethnicity, a waist girth, a systolic blood pressure, and a diastolic blood pressure. The invasive measurement can include at least one of LDL cholesterol, HDL cholesterol, triglycerides (mg/dL), and blood glucose level (fasting blood sugar, mM). From 402, method 400 proceeds to 404.

At 404, vector representations are generated by an unsupervised learning algorithm. The unsupervised learning algorithm can be configured to perform operations including: processing the genetic data to generate groups of term co-occurrence, formatting the groups to generate a matrix, setting soft constraints for each entry of the matrix, adding a weight to each entry of the matrix, and generating the vector representations based on the matrix.

The unsupervised learning algorithm can include a modified version of skip-gram model for Char2Vec embedding for RS numbers, risk alleles and genetic variants. Given a window size of n words around a word w, the skip-gram model can predict the neighboring words given the current word. In some implementations, the unsupervised learning algorithm can include an algorithm like word2vec or a slightly modified version of algorithm with similar functionality. The word2vec tool can include two models: skip-gram and continuous bag of words (CBOW). The CBOW model can predict the current word w, given the neighboring words in the window.

A skip-gram model can be used to determine vectorial representations (vectors) for each word w having a place with a given vocabulary {1 . . . W}. The vectors are prepared to foresee words showing up inside the setting window of a given focus word. The skip-gram model maximizes the probability of a context word given the focus word as described by the following equation:

Σₜ₌₁^{T}Σ_{cϵC}_{ₜ} logp(w_{c}|wₜ), (3)

where Cₜ is the context window, w_{c} is the context word, and wₜ is the focus word.

To calculate the probability of a context word given the focus word is computed as:

$$p ( {w}_{c} \vert {w}_{t} ) = \frac{{e}^{s ( {w}_{t} , {w}_{c} )}}{{Σ}_{j = 1}^{W} {e}^{s ( {w}_{t} , j )}} , \qquad ( 4 )$$

where s (scoring function) is a dot product of wₜ and w_{c}, and

$$\frac{{e}^{s ( {w}_{t} , {w}_{c} )}}{{Σ}_{j = 1}^{W} {e}^{s ( {w}_{t} , j )}}$$

is called the softmax function.

The scoring function for n-grams is given as:

s(w, c)=Σ_{gϵG}_{_{w}}\vec{z}_{g}^{T}\vec{v}_{c}, (5)

where G_{w} is the set of all n-grams present in word w. The term z_{g} denotes the vector associated with the n-gram g, and v_{c} is the vector of the context word c. A word representation is derived from the vectors through their constituent n-grams. From 404, method 400 proceeds to 406.

At 406, health risk features are extracted. In response to generating vectors from the characters, the vectors can be used as an input to a clustering algorithm to extract health risk features. The unsupervised clustering algorithms can be configured to find the outliers based on the RS numbers and base pairs of Risk Alleles.

In some implementations, genetic variants are classified based on chromosome types and associations. For example, CYP1A1 belongs to Chromosome 15. The set of known genotypes (RS numbers) for a given genetic variant can be retrieved. The data collected from a database (e.g., 23andMe) contains the genotypes (RS numbers) mapped to their respective risk alleles. For example, for CYP1A1 the RS numbers include: RS4986883, RS4646903,RS4646422, RS4646421, RS2606345, RS2470893, RS1799814, and RS1048943. The RS numbers can be clustered based on the genetic type to determine the chromosome to which they belong. The genotypes (RS numbers) can be clustered based on their homozygosity and heterozygosity. The retrieved RS numbers for each genetic variant can be clustered as RS numbers versus base pairs (AA, TT, CC, GG) (homozygous) or RS numbers versus base pairs (AT, GT, AC, CG, etc.) (heterozygous).

A k-means algorithm can be used for unsupervised clustering. The k-means algorithm utilizes iterative refinement to generate k-centroids for a dataset of genotypes (RS numbers). The dataset can include a collection of features at each data point. The inputs to the k-means algorithm are the vectorized genetic parameters and the number of clusters K. The clustering can include grouping based on an informational index according to a predetermined k number of clusters. The k-means algorithm can be configured to define a k-number of centroids for each cluster. The k-number of centroids, can either be randomly created or arbitrarily chosen from the data set. The k-number of centroids can be placed apart from each other, to enhance differentiation between normal genetic variant and genetic variant with outliers. The center of each cluster is analyzed to determine which data points belong to that particular cluster. In response to mapping each data point to the respective cluster, k-centroids are re-computed and k-new centroids are generated. In response to generating k-new centroids, data set points are allocated to the new nearest center. The data can be remapped until no more changes are done and the k-new centroids are identical to the previously determined k-centroids, which remain stationary. To minimize the objective function, a squared error function can be used:

J(V)=Σᵢ₌₁^{C}Σⱼ₌₁^{C}^{ᵢ}(∥xᵢ−vⱼ∥),² (6)

where, ∥xᵢ=vⱼ∥ is the Euclidean distance between xᵢ and v_{j.}, cᵢ is the number of data points in i^{th} cluster, and c is the number of cluster centers. An example of a k-means algorithm can include the following steps 1-6.

Step 1: Randomly select c clusters.

Step 2: Determine the distance between each data point and selected clusters.

Step 3: Assigning data points to the cluster center whose distance from cluster center is the minimum relative to all cluster centers.

Step 4: Determining new cluster centers:

vᵢ=(1/Cᵢ)Σⱼ₌₁^{C}^{ᵢ}xᵢ, (7)

where cᵢ is the number of data points in the cluster.

Step 5: Determine the distance between each data point and the center of the new clusters.

Step 6: Determine whether data points were reassigned. In response to determining that one or more data points were reassigned, repeat the process from Step 3, else, stop.

In some implementations, different kinds of clustering algorithms can be used, such as fuzzy k-means, hierarchical agglomerative clustering, or a mixture of Gaussians.

In a fuzzy k-means algorithm each point has a likelihood of having a place with each cluster, as opposed to each point having a place with only one group as it is the situation in the conventional k-means. The fuzzy k-means algorithm can manage the issue where points are to some degree in the middle of points. The fuzzy k-means algorithm can manage the issue where points are generally vague by supplanting separation with likelihood, for example, having likelihood in respect to the converse of the separation.

In a hierarchical agglomerative clustering algorithm bunches of points are recursively consolidated until all clusters have been merged into a single cluster that contains all points. The hierarchical agglomerative clustering algorithm includes the following operations: determining the distance function for items d(xᵢ, xⱼ); selecting a distance function for clusters D(Ci, Cj), such that for clusters formed by just one point, D is reduced to d; and clustering starts from N where each cluster contains one item. In some implementations, the hierarchical agglomerative clustering algorithm includes the following operations: utilizing the present matrix of cluster distances to discover two nearest clusters; refreshing the list of clusters by blending the two nearest clusters; and refreshing the matrix of cluster distances appropriately. The operations can be repeated until all items are in one cluster.

In a mixture of Gaussians algorithm, the clusters are formed by considering Gaussian distributions centered on their barycenters.

A Gaussian function is described by the following equation:

$$N ( x ; μ , {σ}^{2} ) = \frac{1}{\sqrt{2 π {σ}^{2}}} {e}^{- \frac{{( x - μ )}^{2}}{2 {σ}^{2}}} , \qquad ( 8 )$$

where μ is the mean and σ² is the variance.

The mixture of Gaussians algorithm begins by fitting the data to a Gaussian mixture model, where the mean, covariance and modes of probability distribution. The modes are a great way of clustering data points. The maximum of class probability is calculated to find out which mode every data point belongs to:

$$P ( i \vert x ) = \frac{{w}_{i} ℊ ( {μ}_{i} , {Σ}_{i} )}{Σ k {w}_{k} ℊ ( {μ}_{k} , Σ k )} \qquad ( 9 )$$

A maximum of previously determined estimates of class probabilities is used to find a set of parameters, which describes each of the points:

$${argmax}_{μ , \sum , w} P ( μ , \sum , w \vert x ) = {argmax}_{μ , \sum , w} P ( x \vert μ , \sum , w ) P ( μ , \sum , w ) \qquad ( 10 )$$

The likelihood is calculated as:

Q=ΣᵢP(i|x, μ, σ)P(x|μ, Σ) (11)

Once the likelihood is calculated, the data can be clustered.

Labels can be generated for the obtained clusters. Labels can be based on the categories of the clusters. For example, heterozygous—CYP1A1—RS4986883—AA as Category1, and so on. Based on the skew of the data, outliers are identified and categorized. The categories can be described in sentences that can be converted to dense vectors by using a particular algorithm, such as an embedding layer in the convolutional neural network or by using GloVe or Word2Vec algorithms.

GloVe algorithm is an unsupervised learning algorithm for obtaining vector representations of words. The GloVe algorithm can include the following operations: determining word co-occurrence, defining soft constraints for each word pair, and defining a cost function.

The penalties associated with soft constraints can be accounted for by the scalar biases. The following equation accounts for the penalties:

wᵢ^{T}wⱼ+bᵢ+bⱼ=log(Xᵢⱼ),

where wᵢ represents the vector for the main word, wⱼ represents the vector for the context word, and bᵢ, bⱼ, are scalar biases for the main and context words.

The operation of determining word co-occurence is based on insights in a type of word co-ocurrence matrix X Every component Xᵢⱼ of a matrix represents to how frequently word i shows up in setting of word j. For each term, setting terms are searched inside a region characterized by a window_size before and after the term and the decay is defined as being inversely proportional to the offset.

decay=l/offset (12)

Soft constraints are defined for each word pair using the following equation:

wᵢ^{T}wⱼ+bᵢ+bⱼ=log(Xᵢⱼ) (13)

The term wᵢ represents the vector for the main word, wⱼ represents the vector for the context word, bᵢ, bⱼ, are scalar biases for the main and context words.

A cost function can be defined using the following equation.

J=Σᵢ₌₁^{V}Σⱼ₌₁^{V}ƒ(Xᵢⱼ)(wᵢ^{T}wⱼ+bᵢ+bⱼ−log Xᵢⱼ)² (14)

The function ƒ is a weighting function, which helps prevent learning only from a limited set of common word pairs:

$$f ( {X}_{i j} ) = \{ \begin{matrix}( \frac{{X}_{ij}}{{x}_{max}} ) a & if {X}_{ij} < XMAX \\ 1 & otherwise\end{matrix} \qquad ( 15 )$$

The vector representations of the words can be used as input for an algorithm configured to identify properties of genes. The algorithm can be configured to select highlights of the words that are the properties of genes, as opposed to variations of the words. The algorithm can use a machine-learning approach to extract discriminant information from a broad array of features, including genomic data, gene expression, protein interactions, and protein structure. The algorithm includes an iterative architecture as a training process on known gene data and recurring cross-validation that optimizes the whole model. The algorithm can use a multi-layer convolutional neural network for optimization.

Neural networks are systems that are designed based on the biological neural networks of animal brains. In the neural networks, there is no particular task for programming and the algorithm learns from various tasks or learning examples. For example, the neural network can be provided with multiple pictures, some of which include images of dogs and are labelled and tagged as “dog” that enable the network to train in recognize dogs. The training can be performed without any previous knowledge about the dogs, such as having canine teeth, sharp ears, etc. The neural networks contain nodes called neurons. The neurons are configured to interact with each other. The neurons receive signals and can process them. In response to completing a processing operation, the neurons transmit signals to the directly connected neurons.

In some implementations, convolutional neural networks (CNN) can be used. The CNN are a class of feed-forward-artificial-neural-networks that are very effective in classification. The CNN can include one or more convolutional layers followed by fully connected layers. The CNN can be designed to take maximum advantage of the 2D inputs by using tied weights and local connections. The CNN can use a pooling function, such as softmax. In some implementations, the CNN can be substituted by an algorithm like support vector machine (SVM) algorithm or Naive Bayes algorithm.

The SVM is a supervised machine-learning algorithm, which is mostly used for classification. The concept of a maximal-marginal classifier is used to explain the concept of an SVM. Given a set of training data, the SVM training algorithm manufactures a model that allocates new cases to one class or the other, making it a non-probabilistic parallel straight classifier. The SVM algorithm can include the following operations: defining an optimal hyperplane to maximize margins; generating a penalty term for misclassifications; and mapping data to a high dimensional space, where it is less demanding to classify with linear decision surfaces to reformulate issue so data that is certainly to a particular space.

The Naïve Bayes Classifier is designed based on the Bayes theorem. Given a hypothesis H and evidence E, Bayes' theorem states that the relationship between the probability of the hypothesis before getting the evidence P(H) and the probability of the hypothesis after getting the evidence P(H|E) is:

$$P ( H \vert E ) = \frac{P ( E \vert H )}{P ( E )} P ( H ) . \qquad ( 16 )$$

Given a class variable y and a dependent feature vector x₁ through xₙ, Bayes' theorem states the following relationship:

$$P ( 𝓎 \vert {x}_{1} , … , {x}_{n} ) = \frac{P ( 𝓎 ) P ( {x}_{1} , {… x}_{n} \vert 𝓎 )}{P ( {x}_{1} , {… x}_{n} )} \qquad ( 17 )$$

The naive independence assumption defines that:

P(xᵢ| [symbol] , x₁, . . . , xₓ₋₁, xᵢ₊₁, . . . , xₙ)=P(xᵢ| [symbol] ), (18)

for all i, the naive independence assumption can be simplified to

$$P ( 𝓎 \vert {x}_{1} , … , {x}_{n} ) = \frac{P ( 𝓎 ) {Π}_{i = 1}^{n} P ( {x}_{i} \vert 𝓎 )}{P ( {x}_{1} , {…x}_{n} )} \qquad ( 19 )$$

Since P (x₁. . . xₙ) is constant given the input, the following classification rule is used:

$$\begin{matrix}P ( 𝓎 \vert {x}_{1} , … , {x}_{n} ) ∝ P ( 𝓎 ) {\prod }_{i = 1}^{n} P ( {x}_{i} \vert 𝓎 ) \\ ⇓ \\ \hat{𝓎} = argmax P ( 𝓎 ) {\prod }_{i = 1}^{n} P ( {x}_{i} \vert 𝓎 )\end{matrix} \qquad ( 20 )$$

The determined properties of genes can be stored or combined with dietary conditions to be used as input for the recipe recommendation system. From 406, method 400 proceeds to 408.

At 408, dietary conditions are determined based on user preferences, user profile and dietary restrictions. User preferences can be determined from user history and/or a questionnaire that is provided to new users and available for updates for existent users. The questionnaire includes multiple questions about individual health conditions. The questionnaire can provide a selection of a diet from multiple options, such as: vegan, vegetarian, white-meat diet, and red-meat diet. The diet selection can be used to generate a decision tree that is displayed to a user as a questionnaire requesting the user to select the ingredients of choice. From 408, method 400 proceeds to 410.

At 410, the properties of genes and the dietary conditions are used to generate a recipe recommendation using a recommendation system. The recommendation systems can include two types: a content-based recommendation system, a collaborative filtering recommendation system or a combination of the two types.

The content-based recommendation system is based on a description of a recipe and the user preferences. The content-based recommendation system can be designed to work with existing profiles of users. The collaborative filtering recommendation system is designed based on the description of the recipe, ingredients present in them, user recipe rating, and user preferences. The collaborative filtering recommendation system can compare different kinds of recipes to generates similarity scores and generate suggestions based on the similarity scores.

In some implementations, the initial recipe recommendations can be based on the list of ingredients and the time complexity selected by the user through the questionnaire. Each recipe can include a tag indicating the time and the complexity associated to the recipe. For example, the tags can indicate easy recipes (0-10 mins), medium level recipes (10-25 mins), and complex recipes (26+ minutes). The system enables a user to provide a user input including user rating of tested recipes. User preferences can be matched, and similarities between users can be found. The similarities can be used to generate new recommendations. Past data of the user can be continuously used as input for the machine learning algorithms, the current health condition of the user is used to update the recommendations and recipes are matched to other users to update similarities. Similarities are determined based on the assumption that the users rate the recipes implicitly or explicitly and users with similar taste in the past will continue to have similar taste over time, as user preferences do not change significantly over time. User recipe ratings and user similarities can be used to generate a matrix. The matrix can be a n by m matrix consisting of the ratings of n users form recipes. Each element of the matrix (i, j) represents how user A rated item j. Each rating can be an integer from 1-5 (reflecting one-star ratings to five-star ratings) if user i has rated recipe j, and 0 if the user has not rated that particular recipe.

The matrix can be used as input to the recommendation system. The output of the recommendation system is a numerical prediction indicating to what degree a user is expected to like or dislike a particular recipe, along with a list of top-n recommended recipes. The top-n recommended recipes can be determined using a user-based-nearest-neighbor collaborative filtering technique.

According to the user-based-nearest-neighbor collaborative filtering technique it is supposed that a user A has not seen the recipe r. The determination of whether recipe r should or should not be recommend to user A can be based on finding a set of users (nearest neighbors) who liked the same items as user A in the past and who have rated the recipe r and calculating an average rating to predict if user A would like recipe r. Average rating can be calculated for all recipes that user A has not seen before. The best-rated recipe can be recommended to user A. The past user data can be studied to see how the diet has impacted the user's health, and make recommendations of recipes to users with similar health conditions.

For example, for each user who hasn't tried or rated the recipe, a set of recipes is recommended. The recommendation can be generated using a K-nearest-neighbor algorithm. The K-nearest-neighbor algorithm can be selected for computational efficiency for large sets of data and increased accuracy. The K-nearest-neighbor algorithm includes multiple operations: defining the number k, selecting k entries from the database, finding the most common classification, and generating an association between the classification and the new sample.

The parameter k can be defined based on the ingredients in the recipe. For example, recipes containing n or more common ingredients are similar to each other and can belong to the same cluster. The recipes for different types of diet are labelled manually and separated (e.g.: Keto diet, Vegan diet, etc.).

A K-fold cross validation can be used to find the most optimum value of k. The K-fold cross validation includes partition the original training data set into k equal subsets. Each subset is called a fold. The folds can be named ƒ₁, ƒ₂, . . . , ƒₖ. For i=1 to i=k, the fold ƒ₁ is considered the validation set and the remaining k−1 folds form the cross validation set. The accuracy of the recommendation system is estimated by averaging the accuracies derived in all k cases of cross validation. In the k-fold cross validation method, all the entries in the original training data set are used for both training and validation. Each entry is used for validation just once. The K-fold cross validation is repeated until an optimum value of k is found.

The selection of k entries from the database is based on ranking the distances from the new sample. The distance can be an Euclidean distance. If p=(p₁, p₂, . . . , pₙ) and q=(q₁, q₂, . . . , qₙ) are two points in Euclidean n-space, the distance (d) from p to q, or from q to p is given by the Pythagorean formula:

$$d ( p , q ) = d ( q , p ) = \sqrt{{( {q}_{1} - {p}_{1} )}^{2} + {( {q}_{2} - {p}_{2} )}^{2} + ⋯ + {( {q}_{n} - {p}_{n} )}^{2}} = \sqrt{{\sum }_{i = 1}^{n} {( {q}_{i} - {p}_{i} )}^{2}} \qquad ( 21 )$$

The parameters p and q are Euclidean Vectors. The length of the vector can be determined as:

$$\vert \vert p \vert \vert = \sqrt{{p}_{2}^{2} + {p}_{2}^{2} + ⋯ = {p}_{n}^{2} = \sqrt{p \cdot p}} \qquad ( 22 )$$

The shortest distances are considered to correspond to the most common classification of the k entries. The concepts of trigonometry can be used to compute the similarity index between the k entries recipes, such as cosine similarity. The ratings for the recipes can be converted into a 1-dimensional vector with the help of a python script. The cosine similarity is computed based on the angle between the rating vectors. Smaller the angle between the vectors, greater is the similarity. Cosine similarities are included in ranges that are between 1 and −1. The properties of cosine similarity include:

Cosine (0°)=1 for very similar recipes.

Cosine (90°)=0 for dissimilar recipes.

Cosine (180°)=−1 for recipes that are opposite in nature.

By the concept of cosine similarity, it is understood that two vectors have the maximum similarity when the angle between them is 0° and both vectors are oriented in the same direction. Two vectors are considered to be dissimilar to each other when they are orthogonal (angle between them is 90°).

For each recipe j user A has not rated yet, the set of users U who are similar to user A and have rated recipe j are found. For each similar user U, u's rating of recipe j is multiplied by the cosine similarity of user A and user U. The weighted ratings are summed up and divided by the number of users in U to determine a weighted average rating for the recipe j.

The recipes are sorted by their weighted average rankings. The average rankings serve as an estimate for what the user rates each recipe. Recipes with higher average rankings are expected to be more likely favored by the user and are recommend to the user with the highest average rankings.

The cosine similarity can be determined for users with similar health conditions in order to recommend recipes. For example, if one of the users was diagnosed with a cardiovascular risk, the user is recommended to eat recipes containing green vegetables for a certain amount of time until the health condition is improved. Past data of users diagnosed with similar conditions are used to make new recommendations.

The following is an example of how the recommendations are made:

TABLE 2
Depicts an example of a user-recipe rating matrix to generate
recommendations.

| | Recipe1 | Recipe2 | Recipe3 | Recipe4 | Recipe5 |
|---|---|---|---|---|---|
| User A | 5 | 3 | 4 | 4 | ? |
| User B | 5 | 1 | 2 | 3 | 3 |
| User C | 4 | 3 | 4 | 3 | 5 |
| User D | 3 | 3 | 1 | 5 | 4 |
| User E | 1 | 5 | 5 | 2 | 1 |

The similarities between users and recipe ratings are calculated and averaged. The following function is used to make a prediction:

$$pred ( a , p ) = \overline{{r}_{a}} + \frac{Σ b ∈ N s i m ( a , b ) * ( {r}_{b , p} - \overline{{r}_{b} )}}{Σ b ∈ N s i m ( a , b )} , \qquad ( 23 )$$

where a and b are the users, p is the rating provided for a particular recipe, and r_{b,p} is the rating provided by user b for recipe p.

The similarities can be used to calculate the neighbor's ratings for the unseen recipe r and verify whether they are higher or lower than their average. The rating differences are combined using similarity with user A as the weight. The neighbors' bias is added/subtracted from the active user's average and is used as a prediction.

In some implementations, ingredients available to the user at a particular time are used to adapt the recipe recommendation, to provide a better user experience. For example a user input including a list of the groceries the user has in the fridge can be used to filter the recipe recommendations. For example, recipes containing the ingredients can be retrieved from the database. A list of recipes that match closely with the list of ingredients can be generated. The recommended recipes can indicate the match of the ingredients and can report missing ingredients.

In some implementations, ingredient combinations in recipes can be predicted based on a plurality of criteria. For example, it can be presumed that the association between smell, taste, touch, and vision are very significant. All of the factors mentioned above interact with each other and create the perception of flavor in food when the person is eating it. Hence, machine learning strategies are appropriate for foreseeing ingredient combinations. A food-pairing network can be used to predict ingredient combinations that work well with each other. The food-pairing network is expected to predict new combinations of ingredients or give combinations of ingredients that work well together. Recipes from different sources can be collected to generate training data. The training data can be classified and a random forest classifier can be used to differentiate types of cuisines based on the ingredients used in them. The random forest classifier can be configured to predict a personalized cuisine given a list of ingredients. The random forest algorithm uses modified bag-of-words to encode the ingredients in the recipe and to generate spare vector occurrences. For example, a recipe A contains: “Brussel sprouts, green pepper, red chili” and recipe B contains: “green pepper, broccoli, Brussel sprouts.” Lists of ingredients are constructed for the recipes:

List 1={“Brussel sprouts”, “green pepper”, “red chili”}

List 2={“green pepper”, “broccoli”, “Brussel sprouts”}

Each ingredient is given a unique identifier:

List 1={“Brussel sprouts”:1, “green pepper”:2, “red chili”:3}

List 2={“green pepper”:2, “broccoli”:4, “Brussel sprouts”:1}

The random forest algorithm uses as input the lists of ingredients with identifiers to perform operations including: randomly selecting “K” features from total “m” features where k<<m where features refer to the ingredients; calculating the node “d” using the best split point among the “K” features, and splitting the node into daughter nodes using the best split. The operations can be repeated until “l” number of nodes has been reached. In some implementations, the operations can be repeated “n” number times to create “n” number of trees. Using the random forest algorithm, different types of cuisines can be classified based on the ingredients used in them.

In some implementations, a custom term frequency-inverse document frequency (tf-idf) can be used to find ingredients that are unique to each cuisine and identify the cuisine with the highest total score. The goal is to design a scoring model that indicates the cuisine that is most probably associated with an ingredient, and given scores for each ingredient against each cuisine, could predict the cuisine of a recipe. For example, the ingredient “curry powder” is an “indicative” ingredient i that is often associated with cuisine c. A higher tf-idf score of i for a cuisine c means that i occurs more frequently in cuisine c than in any other cuisines. Other ingredients, such as water, salt and pepper, are common across many cuisines, and are not “indicative” ingredients. For each ingredient in the training set, the tf-idf score is calculated with respect to a cuisine type as follows:

$$\begin{matrix}tf ( i , c ) = number of times ingredient i appears in c & ( 24 ) \\ idf ( i , C ) = \frac{number of cuisines in c}{number of cuisines that contain ingredient i} & ( 25 ) \\ tfidf ( i , c , C ) = tf ( i , c ) * idf ( i , C ) . & ( 26 )\end{matrix}$$

The most probable cuisine a recipe belongs to can be identified by using the model to calculate the tf-idf scores for each ingredient with respect to each cuisine and by summing the scores by cuisine. The cuisine with the highest score is identified as being the most probable cuisine associated with a recipe.

score(R, C)=max(Σ_{iϵR}tƒidƒ(i, c, C)∀cϵC) (27)

TABLE 3
shows an example of how tf-idf works.

| | | | |
|---|---|---|---|
| X | 311 | 260 | 0 |
| Y | 0 | 58 | 0 |
| Z | 0 | 12 | 15 |
| Total | 311 | 330 | 15 |

Table 3 illustrates an example of a set of cuisines and ingredients of a recipe that are analyzed to identify the cuisine that is most probably associated with the recipe. The set of cuisines includes A, B, and C, and the model is given a recipe with ingredients X, Y, and Z. The tf-idf scores can be calculated for each ingredient-cuisine pair. The last row includes the sums of the tf-idf scores and indicates that cuisine B has the highest total score for the three ingredients.

In some implementations, matrix decomposition can be used as a first predictive model configured to find good ingredient combinations. In matrix decomposition algorithm, only the ingredient combinations found in a particular recipe are used to predict good flavor combinations. The information found in the flavor data can be added to the model, since ingredients with the same flavor compounds are expected to be compatible with each other. The matrix decomposition algorithm can be initiated by generating a matrix composed of ingredients and recipes.

For example, let A be an m×n matrix. The singular value decomposition (SVD) of A, can be performed using the following equation:

A=UΣV^{T}, (28)

where U is m×m and orthogonal, V is n×n and orthogonal, and Σ is an m×n diagonal matrix with non-negative diagonal entries. Given a convergence criterion ε, a matrix U, defined as U=A can include columns that are left singular vectors, and another matrix V defined as the identity matrix can include columns that are right singular vectors.

For all pairs i<j

$$[ \begin{matrix}∝ & ϒ \\ ϒ & β\end{matrix} ] ≡ ( i , j ) \qquad ( 29 )$$

The submatrix of U^{T}U can be determined considering that:

∝=Σₖ₌₁ⁿUₖᵢ² (30)

β=Σₖ₌₁ⁿUₖⱼ² (31)

γ=Σₖ₌₁ⁿUₖᵢUₖⱼ (32)

A Jacobi rotation can be used to diagonalize

$$[ \begin{matrix}∝ & ϒ \\ ϒ & β\end{matrix} ] .$$

ç=(β−∝)/(2_{γ})

(ç)/(|ç|+{square root over (1+ç²))}

t−signum

c=1/\sqrt{1+t²} (33)

[symbol]

=ct (34)

The columns i and j of U can be updated performing the operations described by the following equations:

for k=1 to n

t=Uₖᵢ (35)

Uₖᵢ=ct−sUₖⱼ (36)

Uₖⱼ=st+cUₖⱼ (37)

The matrix V of right singular vectors can be updated performing the operations described by the following equations.

for k=1 to n

t=Vₖᵢ (38)

Vₖᵢ=ct−sVₖⱼ (39)

Vₖⱼ=st=cVₖⱼ (40)

|c|/\sqrt{∝β≤ϵ} (41)

The computed singular values are the norms of the columns of the final matrix U and the computed left singular vectors are the normalized columns of the final matrix U. The columns of matrix V include the computed right singular vectors. From 410, method 400 proceeds to 412.

At 412, the recipe recommendation can be transmitted for display to a user. After 412, method 400 stops.

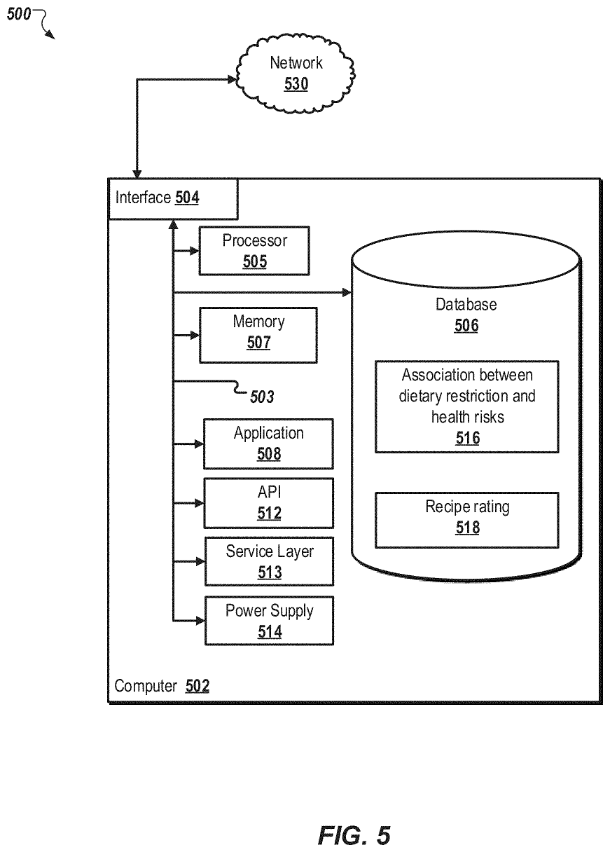

FIG. 5 is a block diagram illustrating an example of a computer system 500 used to provide computational functionalities associated with described algorithms, methods, functions, processes, flows, and procedures, according to an implementation of the present disclosure. The illustrated computer 502 is intended to encompass any computing device such as a server, desktop computer, laptop/notebook computer, wireless data port, smart phone, personal data assistant (PDA), tablet computing device, one or more processors within the devices, another computing device, or a combination of computing devices, including physical or virtual instances of the computing device, or a combination of physical or virtual instances of the computing device. Additionally, the computer 502 can comprise a computer that includes an input device, such as a keypad, keyboard, touch screen, another input device, or a combination of input devices that can accept user information, and an output device that conveys information associated with the operation of the computer 502, including digital data, visual, audio, another type of information, or a combination of types of information, on a graphical-type user interface (UI) (or GUI) or other UI.

The computer 502 can serve in a role in a computer system as a client, network component, a server, a database or another persistency, another role, or a combination of roles for performing the subject matter described in the present disclosure. The illustrated computer 502 is communicably coupled with a network 530. In some implementations, one or more components of the computer 502 can be configured to operate within an environment, including cloud computing based, local, global, another environment, or a combination of environments.

At a high level, the computer 502 is an electronic computing device operable to receive, transmit, process, store, or manage data and information associated with the described subject matter. According to some implementations, the computer 502 can also include or be communicably coupled with a server, including an application server, e-mail server, web server, caching server, streaming data server, another server, or a combination of servers.

The computer 502 can receive requests over network 530 (for example, from a client software application executing on another computer 502) and respond to the received requests by processing the received requests using a software application or a combination of software applications. In addition, requests can also be sent to the computer 502 from internal users (for example, from a command console or by another internal access method), external or third parties, or other entities, individuals, systems, or computers.

Each of the components of the computer 502 can communicate using a system bus 503. In some implementations, any or all of the components of the computer 502, including hardware, software, or a combination of hardware and software, can interface over the system bus 503 using an application-programming interface (API) 512, a service layer 513, or a combination of the API 512 and service layer 513. The API 512 can include particular applications for routines, data structures, and object classes. The API 512 can be either computer-language independent or dependent and refer to a complete interface, a single function, or even a set of APIs. The service layer 513 provides software services to the computer 502 or other components (whether illustrated or not) that are communicably coupled to the computer 502. The functionality of the computer 502 can be accessible for all service consumers using this service layer. Software services, such as those provided by the service layer 513, provide reusable, defined functionalities through a defined interface. For example, the interface can be software written in JAVA, C++, another computing language, or a combination of computing languages providing data in extensible markup language (XML) format, another format, or a combination of formats. While illustrated as an integrated component of the computer 502, alternative implementations can illustrate the API 512 or the service layer 513 as stand-alone components in relation to other components of the computer 502 or other components (whether illustrated or not) that are communicably coupled to the computer 502. Moreover, any or all parts of the API 512 or the service layer 513 can be implemented as a child or a sub-module of another software module, enterprise application, or hardware module without departing from the scope of the present disclosure.

The computer 502 includes an interface 504. Although illustrated as a single interface 504 in FIG. 5, two or more interfaces 504 can be used according to particular needs, desires, or particular implementations of the computer 502. The interface 504 is used by the computer 502 for communicating with another computing system (whether illustrated or not) that is communicatively linked to the network 530 in a distributed environment. Generally, the interface 504 is operable to communicate with the network 530 and comprises logic encoded in software, hardware, or a combination of software and hardware. The interface 504 can comprise software supporting one or more communication protocols associated with communications such that the network 530 or interface's hardware is operable to communicate physical signals within and outside of the illustrated computer 502.

The computer 502 includes a processor 505. Although illustrated as a single processor 505 in FIG. 5, two or more processors can be used according to particular needs, desires, or particular implementations of the computer 502. Generally, the processor 505 executes instructions and manipulates data to perform the operations of the computer 502 and any algorithms, methods, functions, processes, flows, and procedures as described in the present disclosure.

The computer 502 also includes a database 506 that can hold data for the computer 502, another component communicatively linked to the network 530 (whether illustrated or not), or a combination of the computer 502 and another component. For example, database 506 can be an in-memory, conventional, or another type of database storing data consistent with the present disclosure. In some implementations, database 506 can be a combination of two or more different database types (for example, a hybrid in-memory and conventional database) according to particular needs, desires, or particular implementations of the computer 502 and the described functionality. Although illustrated as a single database 506 in FIG. 5, two or more databases of similar or differing types can be used according to particular needs, desires, or particular implementations of the computer 502 and the described functionality. While database 506 is illustrated as an integral component of the computer 502, in alternative implementations, database 506 can be external to the computer 502. As illustrated, the database 506 holds previously described associations between dietary restrictions and health risks 516 and recipe rating 518.

The computer 502 also includes a memory 507 that can hold data for the computer 502, another component or components communicatively linked to the network 530 (whether illustrated or not), or a combination of the computer 502 and another component. Memory 507 can store any data consistent with the present disclosure. In some implementations, memory 507 can be a combination of two or more different types of memory (for example, a combination of semiconductor and magnetic storage) according to particular needs, desires, or particular implementations of the computer 502 and the described functionality. Although illustrated as a single memory 507 in FIG. 5, two or more memories 507 or similar or differing types can be used according to particular needs, desires, or particular implementations of the computer 502 and the described functionality. While memory 507 is illustrated as an integral component of the computer 502, in alternative implementations, memory 507 can be external to the computer 502.

The application 508 is an algorithmic software engine providing functionality according to particular needs, desires, or particular implementations of the computer 502, particularly with respect to functionality described in the present disclosure. For example, application 508 can serve as one or more components, modules, or applications. Further, although illustrated as a single application 508, the application 508 can be implemented as multiple applications 508 on the computer 502. In addition, although illustrated as integral to the computer 502, in alternative implementations, the application 508 can be external to the computer 502.

The computer 502 can also include a power supply 514. The power supply 514 can include a rechargeable or non-rechargeable battery that can be configured to be either user- or non-user-replaceable. In some implementations, the power supply 514 can include power-conversion or management circuits (including recharging, standby, or another power management functionality). In some implementations, the power-supply 514 can include a power plug to allow the computer 502 to be plugged into a wall socket or another power source to, for example, power the computer 502 or recharge a rechargeable battery.

There can be any number of computers 502 associated with, or external to, a computer system containing computer 502, each computer 502 communicating over network 530. Further, the term “client,” “user,” or other appropriate terminology can be used interchangeably, as appropriate, without departing from the scope of the present disclosure. Moreover, the present disclosure contemplates that many users can use one computer 502, or that one user can use multiple computers 502.

Described implementations of the subject matter can include one or more features, alone or in combination.

For example, in a first implementation, a computer-implemented method for generating health recommendations based on user data that is executed by one or more processors includes: receiving genetic data and user data of a user, processing the genetic data to generate vector representations of a fixed size by using an unsupervised learning algorithm, extracting health risk features from the vector representations by using a multi-layer convolutional neural network, determining dietary conditions associated with the health risk features, generating the health recommendation based on the dietary conditions and the user data, and displaying the health recommendation for the user.

Implementations of the subject matter and the functional operations described in the present disclosure can be implemented in digital electronic circuitry, in tangibly embodied computer software or firmware, in computer hardware, including the structures disclosed in the present disclosure and their structural equivalents, or in combinations of one or more of them. Software implementations of the described subject matter can be implemented as one or more computer programs, that is, one or more modules of computer program instructions encoded on a tangible, non-transitory, computer-readable computer-storage medium for execution by, or to control the operation of, data processing apparatus. Alternatively, or additionally, the program instructions can be encoded in/on an artificially generated propagated signal, for example, a machine-generated electrical, optical, or electromagnetic signal that is generated to encode information for transmission to a receiver apparatus for execution by a data processing apparatus. The computer-storage medium can be a machine-readable storage device, a machine-readable storage substrate, a random or serial access memory device, or a combination of computer-storage mediums. Configuring one or more computers means that the one or more computers have installed hardware, firmware, or software (or combinations of hardware, firmware, and software) so that when the software is executed by the one or more computers, particular computing operations are performed.

The term “real-time,” “real time,” “realtime,” “real (fast) time (RFT),” “near(ly) real-time (NRT),” “quasi real-time,” or similar terms (as understood by one of ordinary skill in the art), means that an action and a response are temporally proximate such that an individual perceives the action and the response occurring substantially simultaneously. For example, the time difference for a response to display (or for an initiation of a display) of data following the individual's action to access the data can be less than 1 millisecond (ms), less than 1 second (s), or less than 5 s. While the requested data need not be displayed (or initiated for display) instantaneously, it is displayed (or initiated for display) without any intentional delay, taking into account processing limitations of a described computing system and time required to, for example, gather, accurately measure, analyze, process, store, or transmit the data.

The terms “data processing apparatus,” “computer,” or “electronic computer device” (or equivalent as understood by one of ordinary skill in the art) refer to data processing hardware and encompass all kinds of apparatus, devices, and machines for processing data, including by way of example, a programmable processor, a computer, or multiple processors or computers. The apparatus can also be, or further include special purpose logic circuitry, for example, a central processing unit (CPU), an FPGA (field programmable gate array), or an ASIC (application-particular integrated circuit). In some implementations, the data processing apparatus or special purpose logic circuitry (or a combination of the data processing apparatus or special purpose logic circuitry) can be hardware- or software-based (or a combination of both hardware- and software-based). The apparatus can optionally include code that creates an execution environment for computer programs, for example, code that constitutes processor firmware, a protocol stack, a database management system, an operating system, or a combination of execution environments. The present disclosure contemplates the use of data processing apparatuses with an operating system of some type, for example LINUX, UNIX, WINDOWS, MAC OS, ANDROID, IOS, another operating system, or a combination of operating systems.

A computer program, which can also be referred to or described as a program, software, a software application, a module, a software module, a script, or code can be written in any form of programming language, including compiled or interpreted languages, or declarative or procedural languages, and it can be deployed in any form, including as a stand-alone program or as a module, component, subroutine, or another unit for use in a computing environment. A computer program can, but need not, correspond to a file in a file system. A program can be stored in a portion of a file that holds other programs or data, for example, one or more scripts stored in a markup language document, in a single file dedicated to the program in question, or in multiple coordinated files, for example, files that store one or more modules, sub-programs, or portions of code. A computer program can be deployed to be executed on one computer or on multiple computers that are located at one site or distributed across multiple sites and interconnected by a communication network.

While portions of the programs illustrated in the various figures can be illustrated as individual modules that implement described features and functionality using various objects, methods, or other processes, the programs can instead include a number of sub-modules, third-party services, components, libraries, and such, as appropriate. Conversely, the features and functionality of various components can be combined into single components, as appropriate. Thresholds used to make computational determinations can be statically, dynamically, or both statically and dynamically determined.

Described methods, processes, or logic flows represent one or more examples of functionality consistent with the present disclosure and are not intended to limit the disclosure to the described or illustrated implementations, but to be accorded the widest scope consistent with described principles and features. The described methods, processes, or logic flows can be performed by one or more programmable computers executing one or more computer programs to perform functions by operating on input data and generating output data. The methods, processes, or logic flows can also be performed by, and apparatus can also be implemented as, special purpose logic circuitry, for example, a CPU, an FPGA, or an ASIC.

Computers for the execution of a computer program can be based on general or special purpose microprocessors, both, or another type of CPU. Generally, a CPU will receive instructions and data from and write to a memory. The essential elements of a computer are a CPU, for performing or executing instructions, and one or more memory devices for storing instructions and data. Generally, a computer will also include, or be operatively coupled to, receive data from or transfer data to, or both, one or more mass storage devices for storing data, for example, magnetic, magneto-optical disks, or optical disks. However, a computer need not have such devices. Moreover, a computer can be embedded in another device, for example, a mobile telephone, a personal digital assistant (PDA), a mobile audio or video player, a game console, a global positioning system (GPS) receiver, or a portable memory storage device.

Non-transitory computer-readable media for storing computer program instructions and data can include all forms of permanent/non-permanent or volatile/non-volatile memory, media and memory devices, including by way of example semiconductor memory devices, for example, random access memory (RAM), read-only memory (ROM), phase change memory (PRAM), static random access memory (SRAM), dynamic random access memory (DRAM), erasable programmable read-only memory (EPROM), electrically erasable programmable read-only memory (EEPROM), and flash memory devices; magnetic devices, for example, tape, cartridges, cassettes, internal/removable disks; magneto-optical disks; and optical memory devices, for example, digital video disc (DVD), CD-ROM, DVD+/-R, DVD-RAM, DVD-ROM, HD-DVD, and BLURAY, and other optical memory technologies. The memory can store various objects or data, including caches, classes, frameworks, applications, modules, backup data, jobs, web pages, web page templates, data structures, database tables, repositories storing dynamic information, or other appropriate information including any parameters, variables, algorithms, instructions, rules, constraints, or references. Additionally, the memory can include other appropriate data, such as logs, policies, security or access data, or reporting files. The processor and the memory can be supplemented by, or incorporated in, special purpose logic circuitry.

To provide for interaction with a user, implementations of the subject matter described in the present disclosure can be implemented on a computer having a display device, for example, a CRT (cathode ray tube), LCD (liquid crystal display), LED (Light Emitting Diode), or plasma monitor, for displaying information to the user and a keyboard and a pointing device, for example, a mouse, trackball, or trackpad by which the user can provide input to the computer. Input can also be provided to the computer using a touchscreen, such as a tablet computer surface with pressure sensitivity, a multi-touch screen using capacitive or electric sensing, or another type of touchscreen. Other types of devices can be used to interact with the user. For example, feedback provided to the user can be any form of sensory feedback (such as, visual, auditory, tactile, or a combination of feedback types). Input from the user can be received in any form, including acoustic, speech, or tactile input. In addition, a computer can interact with the user by sending documents to and receiving documents from a client-computing device that is used by the user (for example, by sending web pages to a web browser on a user's mobile computing device in response to requests received from the web browser).

The term “graphical user interface,” or “GUI,” can be used in the singular or the plural to describe one or more graphical user interfaces and each of the displays of a particular graphical user interface. Therefore, a GUI can represent any graphical user interface, including but not limited to, a web browser, a touch screen, or a command line interface (CLI) that processes information and efficiently presents the information results to the user. In general, a GUI can include a plurality of user interface (UI) elements, some or all associated with a web browser, such as interactive fields, pull-down lists, and buttons.

Implementations of the subject matter described in the present disclosure can be implemented in a computing system that includes a back-end component, for example, as a data server, or that includes a middleware component, for example, an application server, or that includes a front-end component, for example, a client computer having a graphical user interface or a Web browser through which a user can interact with an implementation of the subject matter described in the present disclosure, or any combination of one or more such back-end, middleware, or front-end components. The components of the system can be interconnected by any form or medium of wireline or wireless digital data communication (or a combination of data communication), for example, a communication network. Examples of communication networks include a local area network (LAN), a radio access network (RAN), a metropolitan area network (MAN), a wide area network (WAN), Worldwide Interoperability for Microwave Access (WIMAX), a wireless local area network (WLAN) using, for example, 802.11 a/b/g/n or 802.20 (or a combination of 802.11x and 802.20 or other protocols consistent with the present disclosure), all or a portion of the Internet, another communication network, or a combination of communication networks. The communication network can communicate with, for example, Internet Protocol (IP) packets, Frame Relay frames, Asynchronous Transfer Mode (ATM) cells, voice, video, data, or other information between network addresses.

The computing system can include clients and servers. A client and server are generally remote from each other and typically interact through a communication network. The relationship of client and server arises by virtue of computer programs running on the respective computers and having a client-server relationship to each other.

While the present disclosure contains many particular implementation details, the should not be construed as limitations on the scope of any invention or on the scope of what can be claimed, but rather as descriptions of features that can be particular to particular implementations of particular inventions. Certain features that are described in the present disclosure in the context of separate implementations can also be implemented, in combination, in a single implementation. Conversely, various features that are described in the context of a single implementation can also be implemented in multiple implementations, separately, or in any sub-combination. Moreover, although previously described features can be described as acting in certain combinations and even initially claimed as such, one or more features from a claimed combination can, in some cases, be excised from the combination, and the claimed combination can be directed to a sub-combination or variation of a sub-combination.

Particular implementations of the subject matter have been described. Other implementations, alterations, and permutations of the described implementations are within the scope of the following claims as will be apparent to those skilled in the art. While operations are depicted in the drawings or claims in a particular order, this should not be understood as requiring that such operations be performed in the particular order shown or in sequential order, or that all illustrated operations be performed (some operations can be considered optional), to achieve desirable results. In certain circumstances, multitasking or parallel processing (or a combination of multitasking and parallel processing) can be advantageous and performed as deemed appropriate.

Moreover, the separation or integration of various system modules and components in the previously described implementations should not be understood as requiring such separation or integration in all implementations, and it should be understood that the described program components and systems can generally be integrated together in a single software product or packaged into multiple software products.

Accordingly, the previously described example implementations do not define or constrain the present disclosure. Other changes, substitutions, and alterations are also possible without departing from the spirit and scope of the present disclosure.

Furthermore, any claimed implementation is considered to be applicable to at least a computer-implemented method; a non-transitory, computer-readable medium storing computer-readable instructions to perform the computer-implemented method; and a computer system comprising a computer memory interoperably coupled with a hardware processor configured to perform the computer-implemented method or the instructions stored on the non-transitory, computer-readable medium.

What is claimed is:

1. A computer-implemented method for generating a health recommendation, the method being executed by one or more processors and comprising:
receiving, by the one or more processors, genetic data and user data of a user;
processing, by the one or more processors, the genetic data to generate vector representations of a fixed size by using an unsupervised learning algorithm;
extracting, by the one or more processors, health risk features from the vector representations by using a multi-layer convolutional neural network;
determining, by the one or more processors, dietary conditions associated with the health risk features;
generating, by the one or more processors, the health recommendation based on the dietary conditions and the user data; and
displaying, by the one or more processors, the health recommendation for the user.

2. The method of claim 1, wherein the genetic data comprises reference numbers of single nucleotide polymorphisms, risk alleles, and genetic variants.

3. The method of claim 1, wherein the user data comprises one or more of dietary restrictions, a panoramic profile, available ingredients, user preferences, and user recipe rating.

4. The method of claim 3, wherein the panoramic profile comprises a metabolic profile based on at least one of a noninvasive measurement and an invasive measurement.

5. The method of claim 4, wherein the noninvasive measurement comprises at least one of a gender, an ethnicity, a waist girth, a systolic blood pressure, and a diastolic blood pressure.

6. The method of claim 4, wherein the invasive measurement comprises at least one of LDL cholesterol, HDL cholesterol, triglycerides (mg/dL), and blood glucose level (fasting blood sugar, mM).

7. The method of claim 1, wherein the unsupervised learning algorithm is characterized by a model for distributed word representation configured to automatically generate linear structures of the vector representations.

8. The method of claim 1, wherein the unsupervised learning algorithm is configured to perform operations comprising:
processing the genetic data to generate groups of term co-occurrence;
formatting the groups to generate a matrix;
setting soft constraints for each entry of the matrix;
adding a weight to each entry of the matrix; and
generating the vector representations based on the matrix.

9. The method of claim 8, wherein generating the groups of term co-occurrence comprises identifying a plurality of portions of the genetic data and determining a frequency of each portion of the genetic data.

10. The method of claim 8, wherein adding the weight prevents learning only from a limited subset of the plurality of portions of the genetic data that is most common.

11. The method of claim 1, wherein processing the genetic data to generate the vector representations of the fixed size comprises:
classifying the vector representations to generate a plurality of clusters, each cluster comprising one or more data points;
determining a first distance between each data point and a center of each cluster;
assigning the one or more data points to one of the plurality of clusters based on the first distance;
determining a new cluster center for each cluster;
determining a second distance between each data point and the new center of each cluster; and
determining whether any data point is reassigned to a different cluster.

12. The method of claim 1, wherein the multi-layer convolutional neural network comprises a plurality of layers comprising a convolution layer configured to extract health risk features from the vector representations and at least one of a rectified linear unit layer configured to add non-linearity to the health risk features, a pooling layer configured to down sample the health risk features, a dropout layer configured to filter out the noise of the health risk features, and a fully connected layer configured to determine patterns within the health risk features.

13. The method of claim 12, wherein the convolution layer comprises a sliding window function that is applied to the vector representations of the fixed size to generate convolved features that are processed to extract the health risk features.

14. The method of claim 12, wherein the rectified linear unit layer is configured to process the health risk features generated by any of the plurality of layers that are identified as being linear.

15. The method of claim 12, wherein the pooling layer comprises a filter configured to determine a maximum value of the health risk features generated by any of the plurality of layers that are within a window size.

16. The method of claim 12, wherein the dropout layer comprises a noise filter configured to remove a portion of the health risk features generated by any of the plurality of layers that are associated to errors.

17. The method of claim 12, wherein the fully connected layer comprises a connecting function configured to add a weight to each pair connection of the health risk features generated by any of the plurality of layers.

18. The method of claim 1, wherein generating the health recommendation comprises identifying a set of users who previously rated recipes similar to the user and who have rated one of a plurality of recipes matching the dietary conditions of the user.

19. The method of claim 10, wherein the plurality of recipes is classified based on weighted average rankings.

20. The method of claim 10, wherein the set of users who previously rated recipes similar to the user is determined based on calculating cosine similarity for users with the dietary conditions.

21. The method of claim 1, wherein generating the health recommendation comprises an analysis of an impact of past rated recipes on the user data.

22. The method of claim 1, wherein generating the health recommendation comprises using a food-pairing network to predict ingredient combinations that match with each other.

23. A non-transitory computer-readable storage medium coupled to one or more processors and having instructions stored thereon which, when executed by the one or more processors, cause the one or more processors to perform operations for generating a health recommendation, the operations comprising:
receiving genetic data and user data of a user;
processing the genetic data to generate vector representations of a fixed size by using an unsupervised learning algorithm;
extracting health risk features from the vector representations by using a multi-layer convolutional neural network;
determining dietary conditions associated with the health risk features;
generating the health recommendation based on the dietary conditions and the user data; and
displaying the health recommendation for the user.

24. A system, comprising:
a computing device; and
a computer-readable storage device coupled to the computing device and having instructions stored thereon which, when executed by the computing device, cause the computing device to perform operations for generating a health recommendation, the operations comprising:
receiving genetic data and user data of a user,
processing the genetic data to generate vector representations of a fixed size by using an unsupervised learning algorithm,
extracting health risk features from the vector representations by using a multi-layer convolutional neural network,
determining dietary conditions associated with the health risk features,
generating the health recommendation based on the dietary conditions and the user data, and
displaying the health recommendation for the user.
